(19) United States
(12) Patent Application Publication
(10) Pub. No.: US 2008/0161728 A1
(43) Pub. Date: Jul. 3, 2008

(54) Guide Wire and Method of Manufacturing Guide Wire

(76) Inventor: Katsuro Mishima, Yugawara-cho (JP)

Correspondence Address: BUCHANAN, INGERSOLL & ROONEY PC, POST OFFICE BOX 1404, ALEXANDRIA, VA, 22313-1404, US

(21) Appl. No.: 11/964,305

(22) Filed: Dec. 26, 2007

Related U.S. Application Data
(60) Provisional application No. 60/884,690, filed on Jan. 12, 2007.

(30) Foreign Application Priority Data
Dec. 29, 2006 (JP) 2006-356914

(51) Int. Cl. A61M 25/00

(52) U.S. Cl. 600/585

(57) ABSTRACT

A guide wire includes a wire body in which a first wire disposed on the distal side and composed of a first material and a second wire disposed on the proximal side of the first wire and composed of a second material are connected to each other through an intermediate member. The intermediate member includes a distal-side portion composed of the first material, a proximal-side portion composed of the second material, and an intermediate portion located between the distal-side portion and the proximal-side portion and composed of a material including the first material and the second material; and the first wire and the distal-side portion of the intermediate member are joined to each other, and the second wire and the proximal-side portion of the intermediate member are joined to each other.

This application claims priority under 35 U.S.C. § 119(e) to U.S. Provisional Application No. 60/884,690 filed on Jan. 12, 2007, the entire content of which is incorporated herein by reference. This application is also based on and claims priority to Japanese Application No. 2006-356914 filed on Dec. 29, 2006, the entire content of which is incorporated herein.

### TECHNOLOGICAL FIELD

The present invention generally relates to a guide wire and to a method of manufacturing a guide wire.

### BACKGROUND DISCUSSION

Guide wires are used to guide a catheter in treating sites at which open surgeries are difficult or which require minimal invasiveness to the body, for example, PTCA (Percutaneous Transluminal Coronary Angioplasty), or in examination such as cardioangiography. A guide wire used in the PTCA procedure is inserted, with its distal end projecting from the distal end of a balloon catheter, into the vicinity of a target angiostenosis portion together with the balloon catheter, and is operated to guide the distal portion of the balloon catheter into the vicinity of the target angiostenosis portion.

In PTA (Percutaneous Transluminal Angioplasty) also, for opening a stenosis portion (occluded portion) in a peripheral blood vessel such as femoral, iliac, renal and shunt blood vessels, a distal portion of a balloon catheter is guided to the vicinity of an angiostenosis portion by use of a guide wire, like in the PTCA procedure.

Since the blood vessels to which such a treating method is applied are bent in a complicated manner, a guide wire used to insert a balloon catheter into the blood vessel is required to have, for example, appropriate flexibility and resilience against bending, pushability and torque transmission performance (generically called “steerability”) for transmitting an operational force from the proximal portion to the distal side, and further, kink resistance (resistance against sharp bending) and the like.

In an attempt to address such need, U.S. Pat. No. 6,916,386 proposes a guide wire having different flexibility between the distal portion and the proximal portion of a core member.

In addition, U.S. Pat. No. 6,001,068 proposes a guide wire which includes a first wire disposed on the distal side and having flexibility, a second wire disposed on the proximal side and having high rigidity, and a pipe-like connecting member connecting the first wire and the second wire to each other and provided with a slit(s) and a groove(s). In this way, the rigidity of the connecting member is gradually enhanced along the direction from the distal side toward the proximal side.

In such a guide wire, a wire having desired characteristics can be disposed respectively on the distal side and on the proximal side. However, since both wires are connected to each other through a pipe-like connecting member, the joint strength between the wires cannot be enhanced, and a sufficient torque transmission performance is difficult to obtain. Further, from a production standpoint, the operation associated with connecting the wires requires considerable labor.

U.S. Patent Application Publication No. 2004/0260206A1 proposes a guide wire in which a member obtained by placing a powder of a first metallic material and a powder of a second metallic material in a mold so that the contents of the powders vary along the direction from one side toward the other side, followed by sintering, is used as an intermediate material, and a distal-side wire composed of the first metallic material and a proximal-side wire composed of the second material are connected to each other through the intermediate member.

However, even in the case of the just-mentioned intermediate member, a sufficient joint strength is difficult to achieve. Further, the strength of the intermediate member is insufficient, and the strength lacks stability.

### SUMMARY

According to one aspect, a guide wire includes a wire body in which a first wire disposed on the distal side and composed of a first material and a second wire disposed on the proximal side of the first wire and composed of a second material are connected to each other through an intermediate member. The intermediate member includes a distal-side portion composed of the first material, a proximal-side portion composed of the second material, and an intermediate portion located between the distal-side portion and the proximal-side portion, and composed of a material including the first material and the second material. The first wire and the distal-side portion of the intermediate member are joined to each other, and the second wire and the proximal-side portion of the intermediate member are joined to each other. The intermediate member is obtained from a laminate including a first portion in which a plurality of first foils composed of the first material are laminated along the longitudinal direction of the wire body, a second portion in which a plurality of second foils composed of the second material are laminated along the longitudinal direction of the wire body, and a third portion disposed between the first portion and the second portion in which a plurality of third foils composed of the first and second materials are laminated along the longitudinal direction of the wire body. The intermediate member is heated and pressed to mutually bond the foils.

The first material preferably is a Ni—Ti alloy or a metallic material containing Ni and Ti. The second material can be a stainless steel.

The first wire and the distal-side portion of the intermediate member are joined to each other, preferably by welding. The second wire and the proximal-side portion of the intermediate member are joined to each other, preferably by welding or soldering. The welding is preferably conducted by butt resistance welding. The intermediate member can be substantially cylindrical or substantially frustoconical in shape. The intermediate portion of the intermediate member preferably has a portion in which the proportion of the first material is gradually decreased and the proportion of the second material is gradually increased, along the direction from the distal-side portion toward the proximal-side portion. The third portion of the laminate can have a portion in which the number or thickness of the first foil(s) is gradually decreased along the direction from the first portion toward the second portion. The third portion of the laminate preferably has a portion in which the number or thickness of the second foil(s) is gradually increased along the direction from the first portion toward the second portion.

Another aspect involves a method of manufacturing a guide wire having a wire body in which a first wire disposed on the distal side and composed of a first material and a second wire disposed on the proximal side of the first wire and composed of a second material are connected to each other through an intermediate member. The method comprises preparing a laminate comprising a first portion obtained by laminating a plurality of first foils composed of the first material along the longitudinal direction of the wire body, a second portion obtained by laminating a plurality of second foils composed of the second material along the longitudinal direction of the Wire body and a third portion disposed between the first portion and the second portion and obtained by laminating a plurality of third foils made of the first and second materials in a mixture along the longitudinal direction of the wire. The laminate is heated and pressed to mutually bond the foils. The method further involves blanking, from the heated and pressed laminate, an intermediate member including a distal-side portion composed of the first material, a proximal-side portion composed of the second material, and an intermediate portion located between the distal-side portion and the proximal-side portion and composed of a material including the first material and the second material and joining the first wire and the distal-side portion of the intermediate member to each other and joining the second wire and the proximal-side portion of the intermediate member to each other, so as to obtain the wire body.

The first material is preferably a Ni—Ti alloy or a metallic material containing Ni and Ti. The second material preferably is a stainless steel. The joining of the first wire and the distal-side portion of the intermediate member to each other is preferably conducted by welding. The joining of the second wire and the proximal portion of the intermediate member can be conducted by welding or soldering. The welding is preferably conducted by butt resistance welding.

### BRIEF DESCRIPTION OF THE DRAWING FIGURES

The foregoing and additional features will become more apparent from the following detailed description considered with reference to the accompanying drawing figures in which like features are designated by like reference numerals.

FIG. 1 is a longitudinal cross-sectional view of a first embodiment of the guide wire disclosed here.

FIG. 2 is an illustration of an aspect of the method of manufacturing the guide wire shown in FIG. 1 illustrating a laminate used to manufacture the guide wire.

FIG. 3 is a schematic illustration of an aspect of the method of manufacturing the guide wire shown in FIG. 1.

FIG. 4 is an illustration of a laminate used in a second embodiment of the guide wire.

FIG. 5 is a longitudinal cross-sectional view of a third embodiment of the guide wire disclosed here.

### DETAILED DESCRIPTION

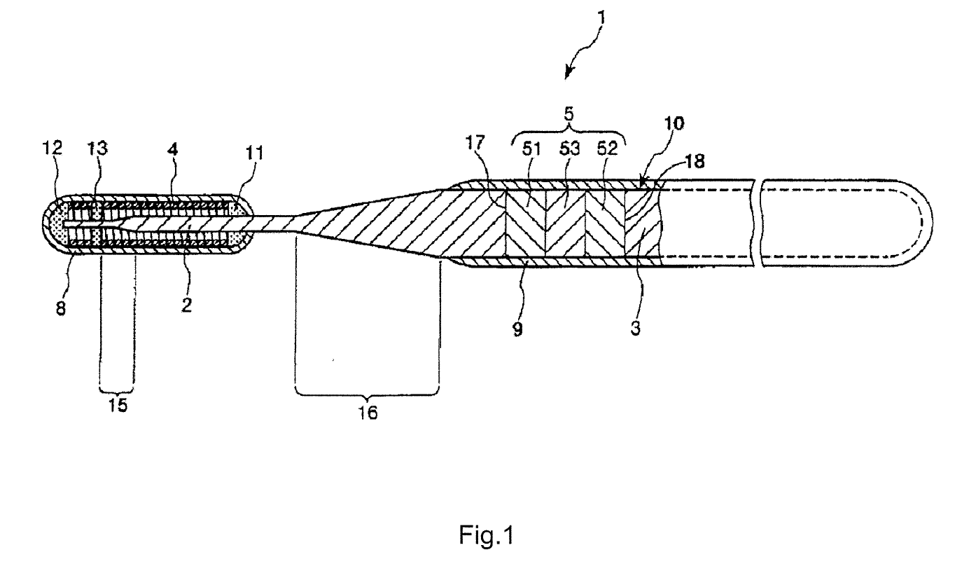

FIG. 1 illustrates in longitudinal cross-section one embodiment of the guide wire disclosed here. For convenience of description, the right side in FIG. 1 is referred to as “proximal”, and the left side is referred to as “distal”. In addition, to help facilitate an understanding, the guide wire shown in FIG. 1 is illustrated as being shortened in the longitudinal direction and exaggerated in the radial (diametrical) direction relative to the actual dimensions of the guide wire. Thus, the ratio between the dimensions in the longitudinal direction and in the radial direction is different from the actual or practical ratio.

The guide wire 1 shown in FIG. 1 is a catheter guide wire having useful application for being inserted in the lumen of a catheter (inclusive of endoscope). The guide wire 1 includes a wire body 10 in which a first wire 2 disposed on the distal side and a second wire 3 disposed on the proximal side of the first wire 2 are connected to each other through an intermediate member 5. In addition, the wire body 10 includes a spiral coil 4. The overall length of the guide wire 1 is preferably about 200 to 5000 mm. In addition, the outer diameter of the guide wire 1 is preferably about 0.2 to 1.2 mm.

The first wire 2 is composed of a flexible or elastic filamentous member. The length of the first wire 2 is preferably about 20 to 1000 mm.

In this embodiment, the first wire 2 includes a portion having a constant outer diameter, and a tapered portion (gradually reduced outer diameter portion) in which the outer diameter is gradually reduced along the distal direction. The latter portion(s) may be provided at only one location or at two or more locations. In the embodiment shown in the drawing figure, the second wire 2 includes two gradually reduced outer diameter portions 15, 16.

By virtue of the gradually reduced outer diameter portions 15, 16, the rigidity (flexural rigidity, torsional rigidity) of the first wire 2 is gradually reduced along the distal direction. As a result, the guide wire 1 possesses good flexibility at its distal portion, whereby trackability related to a blood vessel or the like, and safety, are enhanced. In addition, kinking (sharp bending) and the like can be prevented.

The taper angles (outer diameter reduction rates) of the gradually reduced outer diameter portions 15, 16 may each be constant along the longitudinal direction of the wire body 10 (hereinafter referred to simply as “the longitudinal direction”) or may each be varied along the longitudinal direction at one location or several spaced apart locations. For example, a configuration may be adopted in which a plurality of portions with a comparatively large taper angle (outer diameter reduction rate) and a plurality of portions with a comparatively small taper angle are positioned in an alternating and repeating pattern.

The outer diameter of the proximal-side portion (a portion on the proximal side relative to the gradually reduced outer diameter portion 16) of the first wire 2 is constant over the range to the proximal end of the first wire 2.

In the configuration shown in the figure, the outer diameter of a distal-side portion (a portion on the distal side relative to the gradually reduced outer diameter portion 15) of the first wire 2 is constant over the range to the distal end face of the first wire 2.

The material (first material) constituting or forming the first wire 2 (the blank material of the first wire 2) is not particularly limited, and various metallic materials such as stainless steels can be used. Among these, alloys showing or exhibiting pseudoelasticity (inclusive of superelastic alloys) are preferred. More preferably, superelastic alloys are used. A superelastic alloy is comparatively flexible, has resilience and is less liable to acquire a tendency toward a certain bending. Therefore, with the first wire 2 composed of a superelastic alloy, the guide wire 1 possesses sufficient flexibility and resilience against bending at its distal-side portion, so that trackability in relation to complicatedly curved or bent blood vessels is enhanced, and quite good steerability can be obtained. In addition, the resilience of the first wire 2 inhibits or prevents the first wire 2 from acquiring a tendency toward a certain bending (set) even when the first wire 2 is repeatedly curved or bent. It is thus possible to inhibit or prevent the steerability from being lowered due to a tendency toward a certain bending which might otherwise be acquired by the first wire 2 during use of the guide wire 1.

Possible elastic (superelastic) metals which can be utilized include those elastic metals whose stress-distortion curve by tension has a variety of shapes, and also those elastic metals whose transformation temperature can or cannot be measured notably such as As (austenite start temperature), Af (austenite finish temperature), Ms (martensite start temperature), and Mf (martensite finish temperature) are included. Further, all of those superelastic metals which are deformed (distorted) by a great amount by stress and return to their original shape in response to removal of the stress are included. Thus, the superelastic alloy includes those which exhibit different tensile stress vs. strain curves (i.e., the superelastic alloys which can be used here are not limited to superelastic alloys having a particular tensile stress vs. strain curve), those which have transformation points such as As, Af, Ms, Mf, whether they are measurable clearly or not, and those which are largely deformed (strained) under stresses and return to their original shape upon removal of the stresses.

As a preferred composition of the superelastic alloy, Ni—Ti-based alloys such as a Ni—Ti alloy containing Ni by 49 to 52 atom %, a Cu—Zn alloy containing Zn by 38.5 to 41.5 weight %, Cu—Zn—X alloys (X is at least one of Be, Si, Sn, Al, and Ga) containing X by 1 to 10 weight %, a Ni—Al alloy containing Al by 36 to 38 atom %, and so forth may be used. Among them, the Ni—Ti-based alloys described above are particularly preferable. It is to be noted that the superelastic alloy represented by Ni—Ti-based alloys is excellent also in adhesive property of a coating layer 5 hereinafter described.

The second wire 3 is disposed on the proximal side of the intermediate member 5. The second wire 3 is composed of a flexible or elastic filamentous member. The length of the second wire 3 is not particularly limited, and is preferably about 20 to 4800 mm.

In the configuration shown in FIG. 1, the outer diameter of the second wire 3 is constant (inclusive of substantially constant) along the longitudinal direction, and is equal (inclusive of approximately equal) to the outer diameter of the proximal end of the first wire 2.

The second wire 3 is composed of a material different from the material of the first wire 2. Particularly, the second wire 3 is preferably composed of a material higher in elasticity (Young's modulus (modulus of longitudinal elasticity), modulus of rigidity (modulus of transverse elasticity), bulk modulus) than the material constituting the first wire 2. This helps ensure that the second wire 3 possesses appropriate rigidity (flexural rigidity, torsional rigidity), that the guide wire 1 is relatively high in so-called flexural strength, that the pushability and torque transmission performance of the guide wire 1 are enhanced, and that better insertion steerability can be obtained.

The material (second material) constituting or forming the second wire 3 (the blank material of the second wire 3) is not particularly limited, preferably so long as it is different from the material constituting the first wire 2. For the material forming the second wire 3 various metallic materials such as stainless steels (for example, all SUS steels such as SUS304, SUS303, SUS316, SUS316L, SUS316J1, SUS316J1L, SUS405, SUS430, SUS434, SUS444, SUS429, SUS430F, and SUS302), piano wire, cobalt alloys, and pseudoelastic alloys can be used. Among these metallic materials, preferred are stainless steels and cobalt alloys are preferred, and stainless steels are more preferred. When the second wire 3 is composed of a stainless steel or a cobalt alloy, the guide wire 1 possesses excellent pushability and torque transmission performance.

The intermediate member 5 is disposed between the first wire 2 and the second wire 3. The end portions of the first wire 2 and the intermediate member 5 are joined to each other, and the end portions of the second wire 3 and the intermediate member 5 are joined to each other. More specifically, the end face of the first wire 2 is connected to the end face of the intermediate member 5, and the end face of the second wire 3 is connected to the end face of the intermediate member 5.

The intermediate member 5 is composed of a flexible or elastic material. In the illustrated configuration, the intermediate member 5 is cylindrical (inclusive of substantially cylindrical) in shape, and its outer diameter is equal (inclusive of substantially equal) to the outer diameter of the proximal end of the first wire 2 and the outer diameter of the distal end of the second wire 3.

In addition, the intermediate member 5 includes a distal-side portion 51 composed of the first material (the same material as that of the first wire 2), a proximal-side portion 52 composed of the second material (the same material as that of the second wire 3), and an intermediate portion 53 located between the distal-side portion 51 and the proximal-side portion 52 and composed of a material including the first material and the second material (for example, a mixture of the first material and the second material). In this embodiment, the intermediate member 5, the proximal-side portion 52 and the distal-side portion 51 possess different rigidities (flexural rigidity, torsional rigidity). The rigidity of the proximal-side portion 52 of the intermediate member 5 is the highest, the rigidity of the distal-side portion 51 of the intermediate member 5 is the lowest, and the rigidity (flexural rigidity, torsional rigidity) of the intermediate portion 53 is between the rigidity of the distal-side portion 51 and the rigidity of the proximal-side portion 52. In addition, the rigidity of the distal-side portion 51 of the intermediate member 5 and the rigidity of the proximal portion of the first wire 2 are equal (inclusive of substantially equal), while the rigidity of the proximal-side portion 52 of the intermediate member 5 and the rigidity of the distal portion of the second wire 3 are equal (inclusive of substantially equal).

With respect to the intermediate member 5, the distal-side portion 51 of the intermediate member 5 is joined to the first wire 2, and the proximal-side portion 52 of the intermediate member 5 is joined to the second wire 3.

This configuration helps ensure that the first wire 2 and the distal-side portion 51 of the intermediate member 5 can be relatively easily and firmly joined to each other, and that the second wire 3 and the proximal-side portion 52 of the intermediate member 5 can be relatively easily and firmly joined to each other. In the case of a conventional guide wire which has a weld portion formed by welding a portion composed of a Ni—Ti alloy and a portion composed of a stainless steel, or a weld portion formed by welding a portion composed of a Ni—Ti alloy and a portion composed of a mixture of a Ni—Ti alloy with a stainless steel, or a weld portion formed by welding a portion composed of a stainless steel and a portion composed of a mixture of a Ni—Ti alloy with a stainless steel, a relatively hard and brittle Fe—Ti intermetallic compound is produced upon welding. Hence, the joint strength at the weld portion has been insufficient. On the other hand, in the case of the guide wire 1 in this embodiment, the portions composed of the same material are joined to each other and so the guide wire 1 is not as susceptible to the same problem as associated with the known guide wire. Namely, the first wire 2 and the second wire 3 of the guide wire 1 can be easily and firmly connected to each other through the intermediate member 5.

In addition, the intermediate portion 53 of the intermediate member 5 is composed of a material comprising the first material and the second material (for example, a mixture of the first material with the second material). Therefore, in the wire body 10 of the guide wire 1, the rigidity (flexural rigidity, torsional rigidity) is gradually reduced along the distal direction from an intermediate portion in the longitudinal direction (axial direction). As a result, kink resistance (resistance against sharp bending) is enhanced, and the guide wire 1 can exhibit excellent steerability.

The method for joining the first wire 2 and the distal-side portion 51 of the intermediate member 5 to each other, and the method for joining the second wire 3 and the proximal-side portion 52 of the intermediate member 5 to each other are not particularly limited. It is preferable, however, that the first wire 2 and the distal-side portion 51 are joined to each other by welding, while the second wire 3 and the proximal-side portion 52 are joined to each other by welding or soldering.

As a result, the joint portion 17 between the first wire 2 and the distal-side portion 51 of the intermediate member 5 as well as the joint portion 18 between the second wire 3 and the proximal-side portion 52 of the intermediate member 5 can be provided with a high joint strength by a relatively simple method. In the guide wire 1, therefore, a torsional torque and a pushing-in force exerted from the second wire 3 can be securely transmitted to the first wire 2.

The method by which the first wire 2 and the distal-side portion 51 of the intermediate member 5 are welded to one another, and the method by which the second wire 3 and the proximal-side portion 52 of the intermediate member 5 are welded to one another are not limited to any particular welding method. Examples of welding methods which can be used include frictional welding, laser welding, and butt resistance welding such as upset welding. Among these, butt resistance welding is preferred in view of the comparatively easy process for carrying out the welding and the high joint strength obtained.

The length (length in the longitudinal direction) of the intermediate member 5 is preferably about 0.1 to 5.0 mm, more preferably about 0.5 to 2.0 mm.

The length of the distal-side portion 51 is preferably about 0.1 to 1.0 mm, more preferably 0.2 to 0.5 mm. The length of the proximal-side portion 52 is preferably about 0.1 to 1.0 mm, more preferably about 0.2 to 0.5 mm. The length of the intermediate portion 53 is preferably about 0.1 to 1.5 mm, more preferably about 0.3 to 1.0 mm.

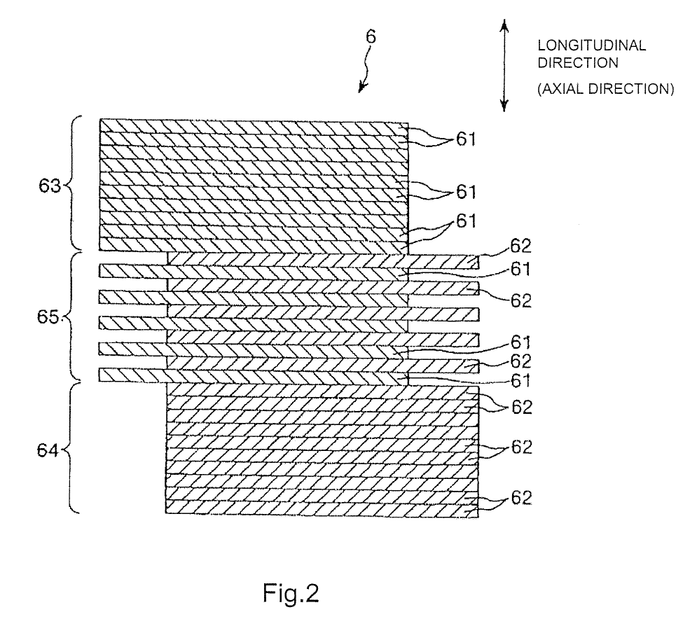

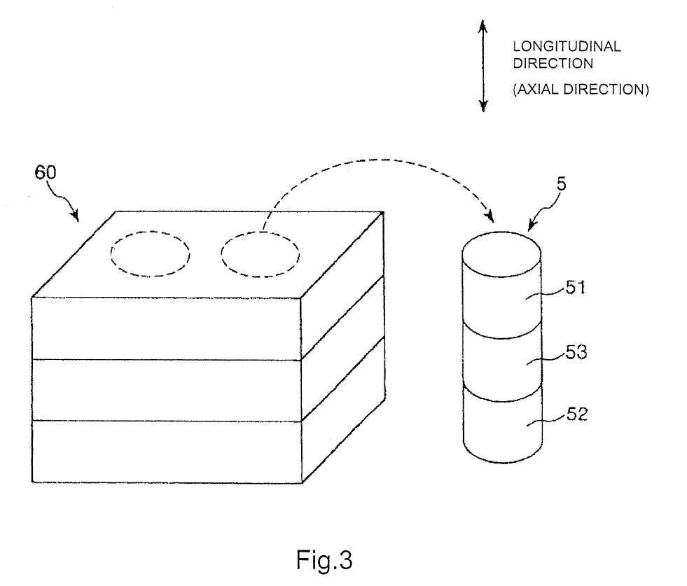

With reference to FIGS. 2 and 3, In this disclosed embodiment, the intermediate member 5 is blanked (obtained) from a block body (metallic foil joint body) 60. This block body is produced by heating and pressing a laminate 6. The laminate includes a first portion 63 obtained by laminating a plurality of first foils (first sheets) 61 composed of the first material (only the first material) along the longitudinal direction, a second portion 64 produced by laminating a plurality of second foils (second sheets) 62 composed of the second material (only the second material) along the longitudinal direction, and a third portion 65 disposed between the first portion 63 and the second portion 64 and produced by laminating a plurality of the first and second foils 61, 62 (interleaved in an alternating sequence or pattern with one another) along the longitudinal direction, so as to mutually bond or (mutually fuse the foils. The third portion can also be described as being produced by laminating a plurality of third plies comprised of the first and second materials along the longitudinal direction. Additional details associated with the method for producing the intermediate member 5 will be described below in connection with description of the method of manufacturing the guide wire 1.

As mentioned above, the coil 4 is disposed at the outer periphery of a distal portion of the first wire 2. The coil 4 is a member obtained by spirally winding a filamentous member (thin wire), and is so disposed as to cover a portion, on at least the distal side, of the first wire 2. In the illustrated embodiment, a distal-side portion of the first wire 2 is positioned centrally in the coil 4 (the distal-side portion of the first wire 2 is positioned in the central portion of the inside of the coil 4). In addition, the distal-side portion of the first wire 2 is located inside the coil 4 such that the outer surface of the first wire 2 is spaced from, and does not contact, the inside surface of the coil 4. The joint portion 17 (i.e., the joint portion 17 between the proximal end face of the first wire 2 and the distal end face of the intermediate member 5) is located on the proximal side relative to the proximal end of the coil 4.

In the embodiment depicted in FIG. 1, in the condition where no external force is exerted on the coil 4, a small gap exists between the adjacent turns of the spirally wound filamentous member forming the coil 4. However, a configuration may be adopted in which the filamentous member is spirally wound closely so that no gap is present between the adjacent turns of the spirally wound filamentous member.

The coil 4 is preferably composed of a metallic material. Examples of the metallic material include not only stainless steels, superelastic alloys, and cobalt alloys, but also noble metals such as gold, platinum, tungsten, etc., and alloys containing such a noble metal (for example, platinum-iridium alloy). Especially where the coils are composed of a radiopaque material such as a noble metal, the guide wire 1 can have a fluoroscopic imageability, so that the guide wire 1 can be inserted into the body while fluoroscopically confirming the position of the distal portion thereof, which is favorable. In addition, the coil 4 may be composed of different materials on the distal side and on the proximal side. For example, a construction may be adopted in which the coil 4 is composed of a coil of a radiopaque material on the distal side, and a coil of a comparatively radiolucent material (e.g., stainless steel) on the proximal side. Though the overall length of the coil 4 (i.e., the axial extent of the coil 4) is not particularly limited, a length of about 5 to 500 mm is preferable.

A proximal portion and a distal portion of the coil 4 are fixed to the first wire 2 by fixing materials 11, 12, respectively. In addition, an intermediate portion of the coil 4 (a portion nearer the distal end than the proximal end) is fixed to the first wire 2 by a fixing material 13. The fixing materials 11, 12, 13 are each composed of solder (brazing filler metal). The fixing materials 11, 12, 13 are not limited to solder, and may be an adhesive. Also, the fixing method for fixing the coil 4 is not limited to the use of a fixing material(s). For example, welding may also be adopted. In addition, to prevent damage to the inside wall of a body lumen such as a blood vessel, the distal surface of the fixing material 12 is preferably rounded in shape.

In this embodiment, with the guide wire being provided with the coil 4 disposed generally as illustrated in FIG. 1, the first wire 2 is covered with the coil 4 and has a small area of contact with the inside wall of a catheter or body lumen, so that sliding resistance is reduced, and the steerability of the guide wire 1 is further enhanced.

Though the coil 4 in the above-described embodiment is formed by use of a filamentous member having a circular cross-sectional shape, the configuration of the coil 4 is not limited in this regard. The cross-sectional shape of the filamentous member may, for example, be an ellipse, a tetragon (particularly, rectangle) or the like.

The outer peripheral surface of the wire body 10 is provided with resin coating layer 8, 9 covering the entire peripheral surface, or a part of the entire outer peripheral surface. In the illustrated embodiment shown in FIG. 1, the resin coating layers 8, 9 are provided on the outer peripheral surfaces of the first wire 2 and the second wire 3, respectively so that each resin coating layer 8, 9 covers only a portion of the longitudinal extent of the outer peripheral surface of the overall wire body. In the illustrated embodiment, the resin cover layers 8, 9 are spaced apart from one another.

The resin coating layers 8, 9 may be provided for any of various purposes. An example is to reduce the friction (sliding resistance) on the guide wire 1 and to enhance slidability, thereby enhancing the steerability of the guide wire 1.

In a variation on the embodiment illustrated in FIG. 1, the resin coating layer 8 or 9 may be provided to cover the outer periphery of the gradually reduced outer diameter portion 16. This makes it possible to further moderate the variation in outer diameter (for example, variation in taper angle) of the wire body 10, and to further enhance the pushability, torque transmission performance and kink resistance of the guide wire 1. It is also possible to enhance the steerability of the guide wire 1 in moving the guide wire along the longitudinal direction.

In order to achieve a reduction in the friction (sliding resistance) of the guide wire 1, the resin coating layers 8, 9 are preferably composed of a friction reducing material as will be described below. This helps ensure that the frictional resistance (sliding resistance) between the guide wire 1 and the inside wall of a catheter used together with the guide wire 1 is reduced, slidability of the guide wire 1 is enhanced, and the steerability of the guide wire 1 in the catheter is enhanced. In addition, since the sliding resistance of the guide wire 1 is lowered, it is possible, when the guide wire 1 is moved and/or rotated in a catheter, to relatively reliably prevent kinking (sharp bending) or torsion of the guide wire 1, particularly kinking or torsion in the vicinity of the joint portions 17, 18.

Examples of material which can be used in the resin coating layers 8, 9 to reduce friction include polyolefins such as polyethylene, polypropylene, etc., polyvinyl chloride, polyesters (PET, PBT, etc.), polyamides, polyimides, polyurethane, polystyrene, polycarbonates, silicone resins, fluororesins (PTFE, ETFE, etc.), and composite materials thereof.

Among these materials, fluroresins (or composite materials containing the same) are particularly favorable as the frictional resistance (sliding resistance) between the guide wire 1 and the inside wall of the catheter can be reduced more effectively, the slidability can be enhanced, and the steerability of the guide wire 1 in the catheter is further improved. In addition, this helps ensure that it is possible, when the guide wire 1 is moved and/or rotated in a catheter, to securely prevent the kinking (sharp bending) or torsion of the guide wire 1, particularly, kinking or torsion in the vicinity of the weld portions.

Where a fluroresin (or a composite material containing the same) is used, coating the wire body 10 with the resin can be conducted keeping the resin material in a heated condition, by such a method as baking and spraying. This promises particularly excellent adhesion between the wire body 10 and the resin coating layers 8, 9.

Where the resin coating layers 8, 9 are each composed of a silicone resin (or a composite material containing the same), it is possible to achieve a relatively assured and firm adhesion of the resin coating layers 8, 9 to the wire body 10 without need for heating at the time of forming the resin coating layers 8, 9 (at the time of coating the wire body 10). More specifically, where the resin coating layers 8, 9 are each composed of a silicone resin (or a composite material containing the same), a reaction-curing type material or the like can be used, so that the formation of the resin coating layers 8, 9 can be carried out at room temperature. With the resin coating layers 8, 9 thus formed at room temperature, the coating can be carried out readily, and the guide wire 1 can be operated (steered) in the condition where a sufficient joint strength at the joint portions 17, 18 is maintained.

The resin coating layers 8, 9 (particularly, the resin coating layer 8 on the distal side) can be provided for the purpose of enhancing safety in inserting the guide wire 1 into a blood vessel or the like. For this purpose, the resin coating layers 8, 9 are preferably composed of a material rich in flexibility (soft material, elastic material).

Examples of the material rich in flexibility include polyolefins such as polyethylene, polypropylene, etc., polyvinyl chloride, polyesters (PET, PBT, etc.), polyamides, polyimides, polyurethane, polystyrene, silicone resins, thermoplastic elastomers such as polyurethane elastomer, polyester elastomers, polyamide elastomers, etc., various rubber materials such as latex rubbers, silicone rubbers, etc., and composite materials obtained by combining two or more of these.

Especially, in the case where the resin coating layers 8, 9 are each composed of one of the above-mentioned thermoplastic elastomers and various rubber materials, the flexibility of a distal portion of the guide wire 1 is further enhanced, so that it is possible at the time of inserting the guide wire 1 into a blood vessel or the like, to relatively securely prevent the guide wire 1 from damaging the blood vessel inside wall or the like, and to realize an extremely high safety.

The resin coating layers 8, 9 may each be a laminate of two or more layers. The resin coating layer 8 and the resin coating layer 9 may be composed of the same material or different materials. For example, the resin coating layer 8 located on the distal side of the guide wire 1 may be composed of the above-mentioned material rich in flexibility (soft material, elastic material), while the resin coating layer 9 located on the proximal side of the guide wire 1 may be composed of the above-mentioned material capable of reducing friction. This makes it possible to achieve simultaneous realization of both enhanced slidability (steerability) and enhanced safety.

The thickness of each of the resin coating layers 8, 9 is not particularly limited, and can be appropriately set in consideration of, for example, the purposes associated with the resin coating layers 8, 9, the materials constituting the resin coating layers 8, 9, the methods of forming the resin coating layers 8, 9. Normally, the resin coating layers 8, 9 preferably each have a thickness (average) of about 1 to 100 μm, more preferably about 1 to 30 μm. If the resin coating layers 8, 9 are too thin, the desired function of forming the resin coating layers 8, 9 may not be displayed sufficiently, and exfoliation of the resin coating layers 8, 9 may occur. On the other hand, if the resin coating layers 8, 9 are too thick, they may exert influences on the physical properties of the wire body 10, and exfoliation of the resin coating layers 8, 9 may occur.

In the guide wire disclosed here, the outer peripheral surface (the surface) of the wire body 10 may be subjected to a treatment (a roughening treatment, a chemical treatment, a heat treatment, or the like) for enhancing adhesion of the resin coating layers 8, 9, or may be provided thereon with an intermediate layer adapted to enhance or facilitate the adhesion of the resin coating layers 8, 9.

The outer surface of at least a distal portion of the guide wire 1 is preferably coated with a hydrophilic material. In this embodiment, the outer peripheral surface of the guide wire 1 in a region ranging from the distal end of the guide wire 1 to the vicinity of the proximal end of the gradually reduced outer diameter portion 16 is coated with a hydrophilic material. This helps ensure that the hydrophilic material produces lubricity upon being wetted so that friction (sliding resistance) on the guide wire 1 is reduced, and the slidability of the guide wire is enhanced. Accordingly, the steerability of the guide wire 1 is improved.

Examples of the hydrophilic material include cellulose based polymer materials, polyethylene oxide based polymer materials, maleic anhydride based polymer materials (for example, maleic acid copolymers such as methyl vinyl ether-maleic anhydride copolymer), acrylamide based polymer materials (for example, polyacrylamide, polyglycidyl methacrylate-dimethylacrylamide (PGMA-DMAA) block copolymer), water-soluble nylon, polyvinyl alcohol, and polyvinyl pyrrolidone.

These hydrophilic materials, in many cases, exhibit lubricity by being wetted (absorbing water) so as to reduce the frictional resistance (sliding resistance) between the guide wire 1 and the inside wall of a catheter used together with the guide wire 1. This enhances the slidability of the guide wire 1, leading to enhanced steerability of the guide wire 1 in a catheter.

An embodiment of the method for manufacturing the guide wire 1 is described below with reference to FIGS. 2 and 3. To help distinguish between the first foils 61 and second foils 62 in FIG. 2, the first foils 61 and the second foils 62 are shown in a staggered condition, and are hatched in opposite inclinations.

1. Preparing (Producing) the Laminate 6

As shown in FIG. 2, a laminate 6 is prepared (produced) which includes a first portion 63 obtained by laminating (i.e., adjacently positioning) a plurality of the first foils (first sheets) 61 composed of a first material along the longitudinal direction (the longitudinal direction of the wire body 10), a second portion 64 obtained by laminating (i.e., adjacently positioning) a plurality of the second foils (second sheets) 62 composed of a second material along the longitudinal direction, and a third portion 65 disposed between the first portion 63 and the second portion 64. The third portion is obtained by laminating (i.e., adjacently positioning) both a plurality of the first foils 61 and a plurality of the second foils 62 along the longitudinal direction. In the laminate 6, the first portion 63 is a portion which will later constitute the distal-side portion 51 of the intermediate member 5, the second portion 64 is a portion which will later constitute the proximal-side portion 52 of the intermediate member 5, and the third portion 65 is a portion which will later constitute the intermediate portion 53 of the intermediate member 5.

More specifically, first, a plurality of the second foils 62 are laminated (adjacently positioned) along the longitudinal direction to form the second portion 64, then a plurality of the first foils 61 and the second foils 62 are laminated thereon as a mixture or in an alternating fashion along the longitudinal direction to form the third portion 65, and a plurality of the first foils 61 are laminated (adjacently positioned) thereon along the longitudinal direction to form the first portion 63. The order in which the first portion 63, the second portion 64 and the third portion 65 are formed is not limited to the just-mentioned order.

In addition, while the first foils 61 and the second foils 62 are alternately laminated or positioned in the third portion 65 in the embodiment shown in FIG. 2, other lamination or positioning patterns may also be adopted.

The thicknesses of the first foil 61 and the second foil 62 are not particularly limited, and they are preferably about 1 to 500 μm, more preferably about 10 to 100 μm.

Here, the first material is preferably a Ni—Ti alloy or a metallic material containing Ni and Ti. For example, in the case where the first material is a Ni—Ti alloy or a metallic material containing Ni and Ti, one first foil 61 may be one foil composed of the Ni—Ti alloy or the material containing Ni and Ti, or may be composed of one or a plurality of Ni foils or Ni alloy foils and one or a plurality of Ti foils or Ti alloy foils. In the latter case, the portion of the first foils 61 is formed to be the Ni—Ti alloy or the metallic material containing Ni and Ti, by the heating and pressing treatment which will be described later.

Instead of the Ni—Ti alloy or the metallic material containing Ni and Ti, the first material can be Ni or Ni alloy. In this case the second material is for example stainless steel.

Instead of stainless steel, the second material can be Ni or Ni alloy. In this case the first material is for example Ni—Ti alloy.

2. Heating and Pressing the Laminate 6 (Heating and Pressing Treatment) (Diffusion Treatment) to Mutually Bond (Fuse) the Foils

The laminate 6 is heated and pressed (heating and pressing treatment), i.e., it is pressed while being heated, so as to mutually bond (fuse) the foils, thereby obtaining (producing) a block body (metallic foil joint body) 60.

The conditions for the heating and pressing treatment (the heating temperature, the pressure exerted on the laminate 6, the treating time, etc.) are not particularly limited, and are appropriately set according to such conditions as the constituent materials of the first and second foils 61, 62, the thicknesses of the first and second foils 61, 62, the number of first and second foils 61, 62, etc. The heating temperature is preferably about 200 to 580° C., more preferably about 350 to 500° C. The pressure applied to the laminate 6 is preferably about 50 to 300 MPa, more preferably about 100 to 200 MPa. The treating time is preferably about 1 to 60 min, more preferably about 10 to 30 min.

By virtue of the heating and pressing treatment, the surfaces of the foils are once melted or mutually diffused, and the adjacent foils are bonded to each other. In this case, oxygen and the oxide constituting an oxide film formed on the surface of each foil are dispersed (diffused) into the inside of the foil, so that the oxide film disappears (is removed). As a result, the block body 60 having a necessary and sufficient strength (high strength) is obtained.

In addition, the heating and pressing treatment is preferably carried out in a reducing atmosphere such as nitrogen gas, ammonia gas, etc. or in a non-oxidizing atmosphere such as at a reduced pressure (in vacuum), etc. This helps ensure that, even when an oxide film is formed on the surface of each foil, the oxide is reduced during the heating and pressing treatment, with the result that the oxide film disappears.

3. Blanking the Intermediate Member 5 from the Block Body 60

As shown in FIG. 3, an intermediate member 5 having a predetermined shape (in the illustrated embodiment, a cylindrical shape) is blanked from the block body 60. In this case, a plurality of the intermediate members 5 can be blanked from one block body 60, whereby a plurality of the intermediate members 5 are obtained or produced.

The method for blanking the intermediate member(s) 5 from the block body 60 is not particularly limited, and various methods can be used. Among the possible methods, a preferred method is one in which blanking is carried out by irradiation with a laser beam.

4. Step of Obtaining (Producing) the Wire Body 10

The proximal portion (the end face on the proximal side) of the first wire 2 and the distal portion (the end face on the distal side) of the distal-side portion 51 of the intermediate member 5 are joined to each other, while the distal portion (the end face on the distal side) of the second wire 3 and the proximal portion (the end face on the proximal side) of the proximal-side portion 52 of the intermediate member 5 are joined to each other. As a result, the first wire 2 and the second wire 3 are connected to each other through the intermediate member 5, and the wire body 10 is obtained or produced.

The method of joining the first wire 2 and the distal-side portion 51 of the intermediate member 5 to each other and the method of joining the second wire 3 and the proximal-side portion 52 of the intermediate member 5 to each other are not particularly limited, and various methods can be used. The joining of the first wire 2 and the distal-side portion 51 to each other is preferably conducted by welding. The joining of the second wire 3 and the proximal-side portion 52 is preferably conducted by welding or soldering.

In addition, the welding methods are not particularly limited. Examples of the welding method include friction welding, laser welding, and butt resistance welding such as upset welding. Among these, butt resistance welding is particularly preferred in view of the comparatively simple process and a high joint strength obtained thereby.

Following the foregoing steps, the wire body 10 is provided with the above-mentioned coil 4, resin coating layers 8, 9 and the like, to obtain the guide wire 1.

As has been described above, the guide wire 1 is comprised of the first wire 2 rich in flexibility on the distal side, and the second wire 3 rich in rigidity on the proximal side. This helps ensure that sufficient flexibility is exhibited on the distal side of the guide wire 1, leading to relatively high safety, and sufficient rigidity is exhibited on the proximal side of the guide wire 1, leading to excellent pushability, torque transmission performance and traceability.

The first wire 2 and the distal-side portion 51 of the intermediate member 5 are composed of the first material (the same material) so that they can be joined to each other relatively easily and firmly. Similarly, the second wire 3 and the proximal-side portion 52 of the intermediate member 5 are composed of the second material (the same material) so that they can be joined to each other relatively easily and firmly. Namely, the first wire 2 and the second wire 3 can be firmly connected to each other through the intermediate member 5.

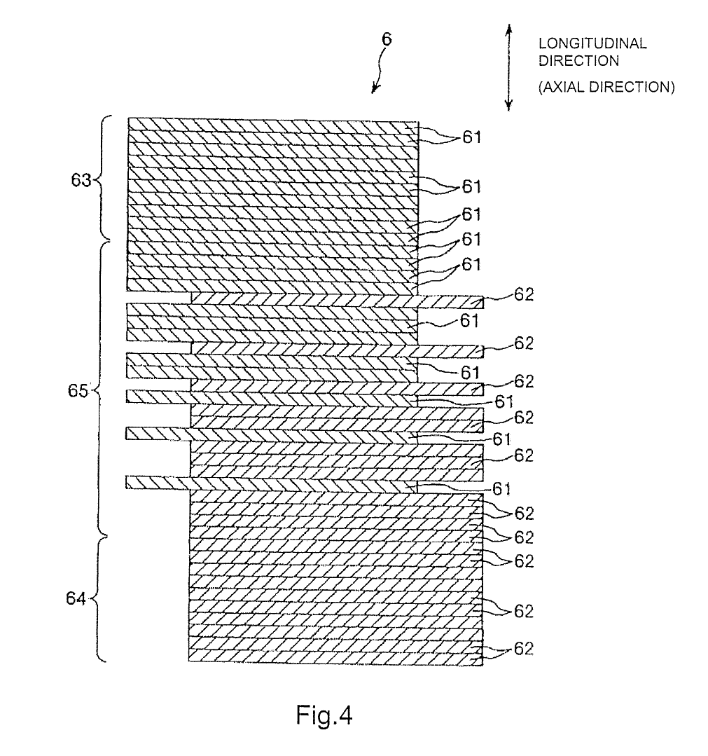

FIG. 4 is a cross-sectional view of a laminate used in a second embodiment of the guide wire disclosed here. The guide wire 1 according to this second embodiment will be described below. The description of this second embodiment of the guide wire 1 will focus primarily on differences relative to the first embodiment. Features in the second embodiment that are the same as those in the first embodiment are identified by the same reference numerals, and a detailed description of such features is not repeated.

The guide wire 1 in the second embodiment is the same as that in the first embodiment above, except for the configuration of the intermediate portion 51 of the intermediate member 5 and the third portion 65 of the laminate 6.

As shown in FIG. 4, in the guide wire 1 according to the second embodiment, a third portion 65 of the laminate 6 comprises a portion in which the number of first foils 61 is gradually decreased in a direction from the first portion 63 toward the second portion 64, and a portion in which the number of second foils 62 is gradually increased in a direction from the first portion 63 toward the second portion 64. More specifically, in the embodiment illustrated in FIG. 4, the number of first foils 61 in the third portion 65 of the laminate 6 gradually decreases moving from the distal end of the third portion 65 to the intermediate portion of the third portion 65, while the number of second foils 62 gradually increases moving from the intermediate portion of the third portion 65 toward the proximal end of the third portion 65.

The intermediate portion 53 of the intermediate member 5 comprises a portion where the proportion of the first material gradually decreases, either stepwise or continuously, in the direction from the distal-side portion 51 toward the proximal-side portion 52. On the other hand, the proportion of the second material gradually increases, either stepwise or continuously, along the direction from the distal-side portion 51 toward the proximal-side portion 52. More specifically, in the illustrated embodiment, the proportion of the first material in the intermediate portion 53 of the intermediate member 5 gradually decreases, either stepwise or continuously, in the direction from the distal end to the proximal end of the intermediate portion 53. On the other hand, the proportion of the second material there is gradually increased either stepwise or continuously as one goes from the distal end to the proximal end of the intermediate portion 53.

In this guide wire 1, the rigidity (flexural rigidity, torsional rigidity) of the intermediate portion 53 of the intermediate member 5 is gradually reduced along the direction from the proximal-side portion 52 toward the distal-side portion 51, whereby kink resistance (resistance against sharp bending) is further enhanced, and better steerability can be obtained.

The third portion 65 of the laminate 6 may have, for example, a portion where the thickness of the first foils 61 is gradually decreased and a portion where the thickness of the second foils 62 is gradually increased, along the direction from the first portion 63 toward the second portion 64, in place of or together with the variations in the numbers of the foils. In other words, by controlling either one or both of the numbers of the foils and the thicknesses of the foils, the proportions of the first material and the second material in the intermediate portion 53 of the intermediate member 5 can be varied along the longitudinal direction.

In addition, this aspect of the guide wire according to the second embodiment is applicable to a third embodiment which will be described below.

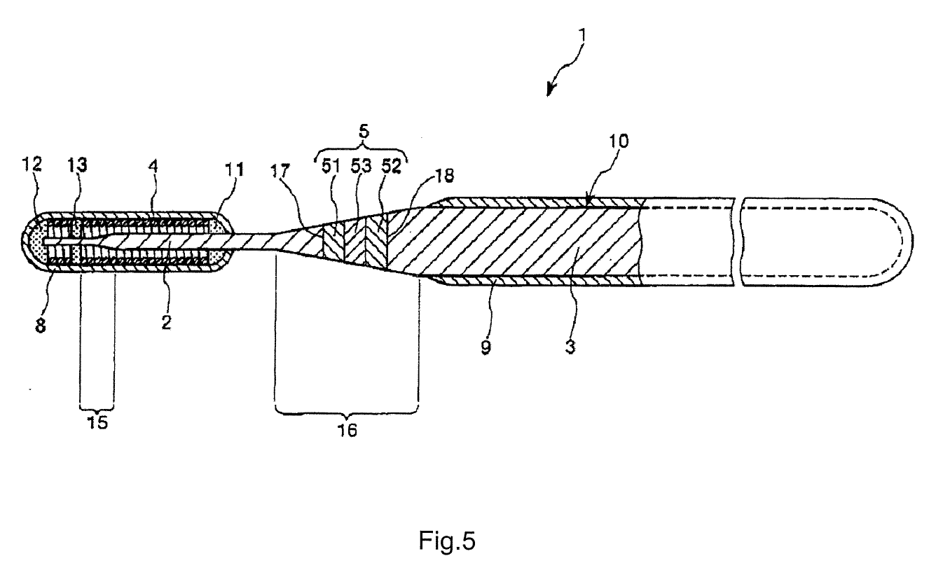

FIG. 5 is a longitudinal cross-sectional view of a third embodiment of the guide wire disclosed here. The guide wire 1 according to this third embodiment are described below. The description of this third embodiment of the guide wire 1 will focus primarily on differences relative to the first embodiment. Features in the third embodiment that are the same as those in the first embodiment are identified by the same reference numerals, and a detailed description of such features is not repeated.

The guide wire 1 in the third embodiment is the same as that in the above-described first embodiment, except for the shape of the intermediate member 5 and the position of the intermediate member 5.

As shown in FIG. 5, in the guide wire 1 according to the third embodiment, the intermediate member 5 is frustoconical (inclusive of substantially frustoconical) in shape, and is disposed in a tapered portion of the wire body 10 where the outer diameter of the wire body 10 is gradually decreased in the distal direction, i.e., in a gradually reduced outer diameter portion 16, thereby constituting a part of the gradually reduced outer diameter portion 16.

While the guide wire here and the method of manufacturing such guide wire have been described above based on the embodiments shown in the drawings, the invention is not limited to the illustrated and described embodiments. For example, the configurations of components can be replaced by other configurations having the same or equivalent functions. In addition, other structures or steps may be added. Additionally, the guide wire can be embodied by combining two or more configurations (characteristics or features) of the above-described embodiments.

Further, the coil 4 may be omitted. In such a case, a filler (particles) of a contrast material (the above-mentioned radiopaque material or the like) may be dispersed in the resin coating layer 8, to constitute the imageable portion.

The principles, preferred embodiment and other disclosed aspects of the guide wire and manufacturing method have been described in the foregoing specification. However, the invention which is intended to be protected is not to be construed as limited to the particular embodiment and variations disclosed. Further, the embodiment described herein is to be regarded as illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the claims, be embraced thereby.

What is claimed is:

1. A guide wire comprising:
a wire body comprised of a first wire disposed on a distal side and a second wire disposed on a proximal side of the first wire;
the first and second wires being connected to each other through an intermediate member;
the first wire being comprised of a first material;
the second wire being comprised of a second material different from the first material;
the intermediate member comprising a distal-side portion comprised of the first material, a proximal-side portion comprised of the second material, and an intermediate portion located between the distal-side portion and the proximal-side portion and comprised of a material including both the first material and the second material; and
the first wire and the distal-side portion of the intermediate member being joined to each other;
the second wire and the proximal-side portion of the intermediate member being joined to each other; and
wherein the intermediate member is a laminate comprising:
a first portion comprised of a plurality of first foils composed of the first material and laminated along the longitudinal direction of the wire body;
a second portion comprised of a plurality of second foils composed of the second material and laminated along the longitudinal direction of the wire body; and
a third portion disposed between the first portion and the second portion and comprised of a plurality of third laminates composed of the first material and the second material and laminated along the longitudinal direction of the wire body;
wherein the laminate of the intermediate member is heated and pressed to mutually bond the first, second and third foils.

2. The guide wire as set forth in claim 1, wherein the first material is a Ni—Ti alloy or a metallic material containing Ni and Ti.

3. The guide wire as set forth in claim 1, wherein the second material is a stainless steel.

4. The guide wire as set forth in claim 1, wherein the first wire and the distal-side portion of the intermediate member are joined to each other by welding.

5. The guide wire as set forth in claim 1, wherein the second wire and the proximal-side portion of the intermediate member are joined to each other by welding or soldering.

6. The guide wire as set forth in claim 4, wherein the welding is conducted by butt resistance welding.

7. The guide wire as set forth in claim 1, wherein the intermediate member is cylindrical or frustoconical in shape.

8. The guide wire as set forth in claim 1, wherein the intermediate portion of the intermediate member has a portion in which a proportion of the first material is gradually decreased and the proportion of the second material is gradually increased, along the direction from the distal-side portion toward the proximal-side portion.

9. The guide wire as set forth in claim 1, wherein the third portion of the laminate has a portion in which a number of the first foil(s) or a thickness of the first foil(s) is gradually decreased along the direction from the first portion toward the second portion.

10. The guide wire as set forth in claim 1, wherein the third portion of the laminate has a portion in which a number of the second foil(s) or a thickness of the second foil(s) is gradually increased along the direction from the first portion toward the second portion.

11. A method of manufacturing a guide wire comprising a wire body in which a first wire is disposed on a distal side, a second wire is disposed on a proximal side of the first wire, the method comprising:
producing a laminate by:
laminating a plurality of first foils comprised of a first material along a longitudinal direction to form a first portion;
laminating a plurality of second foils comprised of a second material along the longitudinal direction to form a second portion;
laminating a plurality of third foils along the longitudinal direction to form a third portion, some of the third foils being comprised of the first material and some of the third foils being comprised of the second material; and
the third portion being positioned between the first portion and the second portion;
heating and pressing the laminate to mutually bond the first, second and third foils to produce a heated and pressed laminate;
blanking, from the heated and pressed laminate, an intermediate member comprising a distal-side portion composed of the first material, a proximal-side portion composed of the second material, and an intermediate portion located between the distal-side portion and the proximal-side portion and composed of a material comprising both the first material and the second material; and
joining the first wire and the distal-side portion of the intermediate member to each other, and joining the second wire and the proximal-side portion of the intermediate member to each other to produce the wire body.

12. The method of manufacturing a guide wire as set forth in claim 11, wherein the first material is a Ni—Ti alloy or a metallic material containing Ni and Ti.

13. The method of manufacturing a guide wire as set forth in claim 11, wherein the second material is a stainless steel.

14. The method of manufacturing a guide wire as set forth in claim 11, wherein the joining of the first wire and the distal-side portion of the intermediate member to each other comprises welding the first wire and the distal-side portion of the intermediate member to each.

15. The method of manufacturing a guide wire as set forth in claim 11, wherein the joining of the second wire and the proximal portion of the intermediate member to each other comprises welding or soldering the second wire and the proximal portion of the intermediate member to each other.

16. The method of manufacturing a guide wire as set forth in claim 15, wherein the welding is conducted by butt resistance welding.

17. The method of manufacturing a guide wire as set forth in claim 11, wherein the laminating of the plurality of third foils to produce the third portion comprises laminating a greater number of third foils made of the first material at one end of the third portion than an opposite end of the third portion, and laminating a greater number of third foils made of the second material at the opposite end of the third portion than the one end of the third portion.

18. The method of manufacturing a guide wire as set forth in claim 11, wherein the laminating of the plurality of third foils to produce the third portion comprises the third foils composed of the first material being alternated with the third foils composed of the second material.

19. The method of manufacturing a guide wire as set forth in claim 11, wherein the blanking of the intermediate member comprises blanking a frustoconically-shaped or cylindrically shaped intermediate member.
